(19) United States
(12) Patent Application Publication
(10) Pub. No.: US 2020/0327976 A1
(43) Pub. Date: Oct. 15, 2020

(54) INTELLIGENT MEDICATION DELIVERY SYSTEMS AND METHODS OF USING A HEALTH MANAGEMENT SOFTWARE APPLICATION

(71) Applicant: Companion Medical, Inc., San Diego, CA (US)

(72) Inventor: Jack PRYOR, San Diego, CA (US)

(21) Appl. No.: 16/847,339

(22) Filed: Apr. 13, 2020

Related U.S. Application Data
(60) Provisional application No. 62/832,814, filed on Apr. 11, 2019.

(51) Int. Cl. G16H 20/60; G16H 20/17; G16H 40/67; G06K 9/00; G06Q 30/06

(52) U.S. Cl. CPC G16H 20/60; G16H 20/17; G06K 2209/17; G06K 9/00671; G06Q 30/0623; G16H 40/67

(57) ABSTRACT

Systems, devices and methods are disclosed for augmented reality and contextual dose calculations for patient users managing a health condition, such as diabetes, using an interactive health management software application on a companion computing device configured to aggregate, process, and display data and command functionalities of the companion device. Implementations of the disclosed methods and systems create an augmented reality experience operated on a companion computing device with the patient user's medical device to simplify carbohydrate estimation and conveniently inform the patient users about food nutrition before they make a selection.

### CROSS-REFERENCES TO RELATED APPLICATIONS

This patent document claims priorities to and benefits of U.S. Provisional Patent Application No. 62/832,814, entitled “AUGMENTED CONTEXT AND AUGMENTED REALITY DOSE CALCULATIONS” and filed on Apr. 11, 2019. The entire content of the aforementioned patent application is incorporated by reference as part of the disclosure of this patent document for all purposes

### TECHNICAL FIELD

The present disclosure relates to medicine administering and tracking systems, devices, and methods.

### BACKGROUND

Diabetes mellitus, also referred to as diabetes, is a metabolic disease associated with high blood sugar due to insufficient production or use of insulin by the body. Diabetes is widely-spread globally, affecting hundreds of millions of people, and is among the leading causes of deaths globally. Diabetes has been categorized into three categories or types: type 1, type 2, and gestational diabetes. Type 1 diabetes is associated with the body's failure to produce sufficient levels of insulin for cells to uptake glucose. Type 2 diabetes is associated with insulin resistance, in which cells fail to use insulin properly. The third type of diabetes is commonly referred to as gestational diabetes, which can occur during pregnancy when a pregnant woman develops a high blood glucose level. Gestational diabetes can develop into type 2 diabetes, but often resolves after the pregnancy.

Various diseases and medical conditions, such as diabetes, require a patient to self-administer doses of a fluid medication. Typically, when administering a fluid medication, the appropriate dose amount is set and dispensed by the patient using a syringe, a pen, or a pump. For example, self-administered medicaments or medicine include insulin used to treat diabetes, Follistim® used to treat infertility, or other injectable medicines such as Humira®, Enbrel®, Lovenox® and Ovidrel®, or others.

### SUMMARY

Systems, devices, and techniques are disclosed for administering medicine to patients and providing health management capabilities for patients and caregivers using a software application and associated medical device, such as a medicine dispensing device like an injection pen, pump, or wearable patch for delivering a prescription drug.

In some aspects, a smart medicine dispensing device (e.g., smart insulin pen) is configured to be in communication with a patient's companion device (e.g., smartphone) having a health management app that serves the patient as a complimentary medical device to the smart medicine dispensing device, in which the health management app is only fully operable based on a specific device pairing with the smart medicine dispensing device to unlock the capabilities intended only to be utilized by a particular user.

In some embodiments, the health management app operates as a complimentary medical device to the medicine dispensing device (also referred to as the pen device), such as by providing various support tools including a dose calculator and decision support modules to calculate and recommend the dose of a medicine (e.g., insulin) the patient should administer using the wirelessly connected medicine dispensing device, as well as to provide control over several functionalities of the dispensing device, e.g., such as monitoring and recording dose sizes dialed on and/or dispensed from the dispensing device. For example, communication between the pen device and the companion device provides the ability for dose tracking, logging, calculating, recommending, and/or communicating dose data with a user (e.g., patient user, health care provider (HCP) and/or caregiver), and other advantages of the intelligent medicine administering system. For example, each bolus that is dispensed by the pen device can be automatically logged and communicated to the companion device.

In some embodiments, the health management software application includes an augmented reality and contextual dose calculation module to calculate or estimate the carb value (e.g. number of grams of carbohydrate) of certain food and/or mealtime insulin needed upon consumption of said food. For example, the app commands the camera of the smartphone and may overlay new data calculated from aggregated data of the user's meal options (based on location, past meal and other information) and recommended insulin dose presently or in the future (based on tracked glucose and insulin data). In some implementations, the app can display estimated grams of carbs, or the equivalent dose of insulin needed for the particular user based on their clinical parameters, where a very high-carb (or high-insulin-required) items may be highlighted, and/or the healthiest or lowest-carb (low-insulin-required) items may be highlighted to help guide selection. Artificial intelligence may be implemented in any number of aspects of the app for any number of reasons, including (but not limited to) optimizing insulin recommendations and/or data aggregation.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIG. 1 is a block diagram representing an example of an intelligent medicine administering system according to one embodiment of the disclosure;

FIG. 2 is a block diagram representing an example of a pen device forming part of the intelligent medicine administering system of FIG. 1;

FIG. 3 is a block diagram representing an example of the companion device of the intelligent medicine administering system of FIG. 1;

FIG. 4 is a block diagram representing an example of software architecture of data processing modules in accordance with some embodiments of the health management app of the intelligent medicine administrating system of FIG. 1;

FIGS. 5-7 show display views of an example implementation of the health management app of the intelligent medicine administering system of FIG. 1 to create an augmented reality experience according to one embodiment of the disclosure;

FIGS. 8-9 show display views of an example implementation of the health management app of the intelligent medicine administering system of FIG. 1 to create an augmented reality experience for a user's refrigerator, pantry or other food storage location;

FIGS. 10-12 show display views of an example implementation of the health management app of the intelligent medicine administering system of FIG. 1 to create an augmented reality experience for a user using food ordering information;

FIGS. 13-15 show display views of an example implementation of the health management app of the intelligent medicine administering system of FIG. 1 to create an augmented reality experience for a user using location information of the user and/or user content; and

FIG. 16-17 show display views of an example implementation of the health management app of the intelligent medicine administering system of FIG. 1 to create an augmented reality experience for a user using prepackaged food information.

### DETAILED DESCRIPTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The intelligent medication delivery system and related methods disclosed herein boasts a variety of features and components that warrant patent protection, both individually and in combination.

A medicament pen is a device that can be used to inject a quantity of a medicine (e.g., single or multiple boluses or doses of the medicine) into a user's body, where more than one dose can be stored in a medicine cartridge contained in the pen device. Pens offer the benefit of simplicity over other methods of delivery, such as syringe or pump-based methods. For example, syringes typically require more steps to deliver a dose, and pumps typically are more complicated to use and require a constant tether to the patient. However, previously there has not been an automated way to track and communicate the doses given with the pen in a simple, effective and reliable manner. In addition, it can be difficult to know how much to dose with the pen, when to dose, or if the patient dosed at all.

As with the dosing of any medication, it is sometimes hard for a patient to remember if a dose has been given. For this reason, for example, pill reminders have been developed where the patient places the medication for the day in a cup labeled with that day. Once they take their medication, there is no question it has been taken because the pills are no longer in the cup. Yet, there are no widely acceptable solutions that address this problem for injection-based therapies. Therefore, without simple, effective and reliable ways of tracking medicine doses, particularly for managing lifelong or chronic conditions like diabetes, patients may easily miss a dose or administer an incorrect dose (e.g., under-dose or overdose) of their medicine which may result in serious, dangerous consequences to their health.

Some medical device systems include independent, standalone software applications (or “apps”) that operate on a computing device. One example includes mobile device software applications that help the users manage their own health and wellness, including dealing with an acute or chronic disease or dysfunction and managing data associated with such uses.

A typical insulin bolus (dose) calculator works by evaluating a diabetic person's (also referred to as “user” or “patient”) current blood glucose level (BG), the insulin in their body from previous doses (e.g., insulin on board or JOB), and the number of grams of carbohydrates (“carbs”) the user is or recently has been eating. From these values and the patient's pre-set clinical parameters, a conventional dose calculator computes an estimated amount of fast-acting insulin to take, based on clinically-validated established equations.

While BG can be measured directly and IOB can be calculated explicitly based on recent insulin doses, the grams of carbs must be manually estimated by the user. This leads to inaccuracy, difficulty in training, and some users being unwilling or unable to use a dose calculator. Yet, for patients, using a dose calculator is much more accurate than the patient roughly estimating or guessing at the proper dose; as such, a dose calculator can contribute to better glycemic control, better safety, and better health. For these reasons, it is desirable for a dose calculator to be used even by people who cannot or will not estimate carbs.

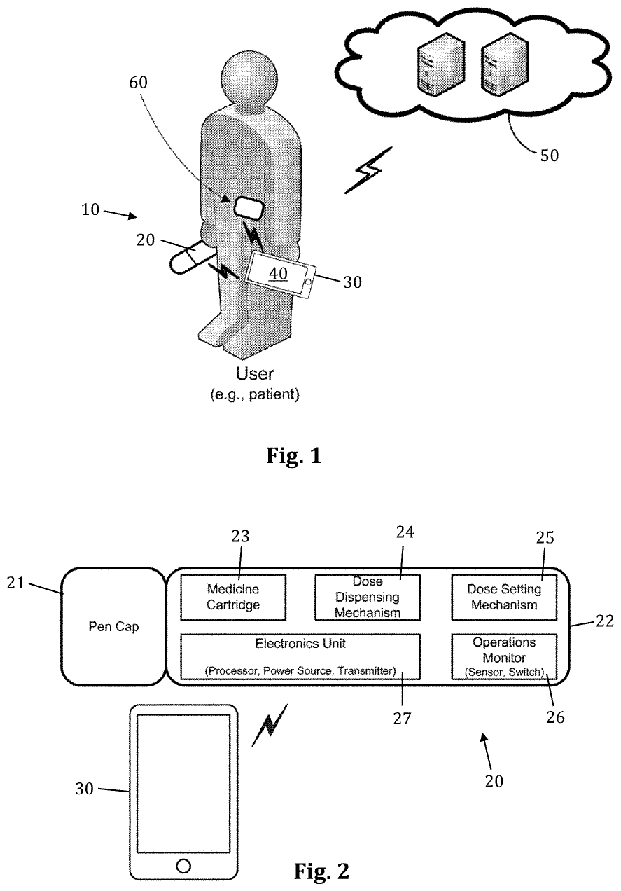

FIG. 1 shows a diagram of an example embodiment of an intelligent medicine administering system 10 in accordance with the present technology. The system 10 includes a pen device 20 in wireless communication with a mobile computing and communication device 30 of a patient user, also referred to as the user's companion device. The pen device 20 is operable to select, set and/or dispense a dose of the medicine for dispensing. In some implementations, the companion device 30 includes a smart phone, tablet, and/or wearable computing device, such as a smart watch, smart glasses, etc. In some implementations, the companion device 30 is in communication with other computing devices, such as a laptop and/or desktop computer, a smart television, or network-based server computer. The companion device 30 includes a health management app 40 that may be associated with the pen device 20 and other smart health management accessories/devices of the intelligent medicine administering system 10.

In some embodiments, the system 10 includes a data processing system 50 in communication with the companion device 30 and/or the pen device 20. The data processing system 50 can include one or more computing devices in a computer system or communication network accessible via the Internet (also referred to as “the cloud”), e.g., including servers and/or databases in the cloud.

The health management app 40 is paired with the pen device 20, which may be a prescription-only medical device. In some implementations, the pairing (also referred to as bonding) of the companion device 30 to the pen device 20 indicates to the health management application 40 that the user is ready to use all features of the application, which can augment performance and provide important features to the intelligent medicine administering system 10. Thus in some implementations the act of pairing (bonding) therefore enables the full functionality of the health management app 40. For example, in some cases the pairing may enable the entire app 40, in which at least a portion of the health management app 40 may be disabled without the specialized pairing; whereas in other cases, the pairing may enable certain features of the health management app 40, which otherwise provides some limited features without the specialized pairing.

In some implementations, for example, the health management app 40 can monitor and/or control functionalities of the pen device 20 and provide a dose calculator and/or decision support modules that can calculate and recommend a dose of the medicine for the patient user to administer using the pen device 20.

The companion device 30 can be used to obtain, process and/or display contextual data that can be used to relate to the patient user's health condition, including the condition for which the pen device 20 is used to treat. In an illustrative example, the companion device 30 is operable to track the patient user's location; the patient user's physical activity including step count, movement distance and/or intensity, estimated calories burned, and/or activity duration; and/or the patient user's interaction pattern with the companion device 30. In some implementations, the app 40 can aggregate and process the contextual data to generate decision support outputs to guide and aid the patient user in using the pen device 20 and/or managing their behavior to promote better health outcomes in treating his/her health condition.

In some embodiments, for example, the system 10 can optionally include a sensor device 60 to monitor one or more health metrics of the patient user. Examples of health metric data monitored by the sensor device 60 include analytes, such as glucose, heart rate, blood pressure, user movement, or other. In some implementations, the sensor device 60 is a wearable sensor device such as a continuous glucose monitor (CGM) to obtain transcutaneous or blood glucose measurements that are processed to produce continuous glucose values. For example, the continuous glucose monitor can include a glucose processing module implemented on a stand-alone display device and/or implemented on the companion device 30, which processes, stores and displays the continuous glucose values for the patient user.

FIG. 2 shows a diagram of an example embodiment of the pen device 20 of the intelligent medicine administering system 10. The pen device 20 is structured to have a cap 21 configured to protect a medicine dispensing element (e.g. needle) and a body 22 configured to contain the medicine cartridge 23 (e.g., which can be replaceable). The pen device 20 is structured to include a dose dispensing mechanism 24 to dispense (e.g., deliver) the medicine contained in the medicine cartridge 23 out of the pen device 20; a dose setting mechanism 25 to select and/or set the dose to be dispensed; an operations monitoring mechanism 26 to determine that the pen device 20 is being operated and/or to monitor the operation of the dose being dispensed (e.g., such as a switch and/or sensor, or an encoder); and an electronics unit 27 that can include a processor, a memory, a battery or other power source, and a transmitter.

The pen device 20 is configured in communication with the patient user's mobile computing and communication device 30, e.g., such as the user's smart phone, tablet, and/or wearable computing device, such as a smart watch, smart glasses, etc. and/or a user's laptop and/or desktop computer, a smart television, or network-based server computer.

The health management app 40 of the companion device 30 associated with the pen device 20 provides a user interface to allow the user to manage his/her health-related data. In some implementations, for example, the health management app 40 can be configured to control some functionalities of the pen device 20. In some implementations, for example, the health management app 40 provides an interactive user interface to allow a user to manage settings of the pen device 20, and settings for the companion device 30 (e.g., smart phone, tablet, or wearable computing device) that can affect the functionality of the system 10. In implementations, for example, the companion device 30 is an independent portable device that the user may carry on his/her person. In example embodiments of the independent portable companion device 30, the companion device 30 includes a data processing unit, wireless communication unit to allow the device to communicate with the pen device 20, and a display unit.

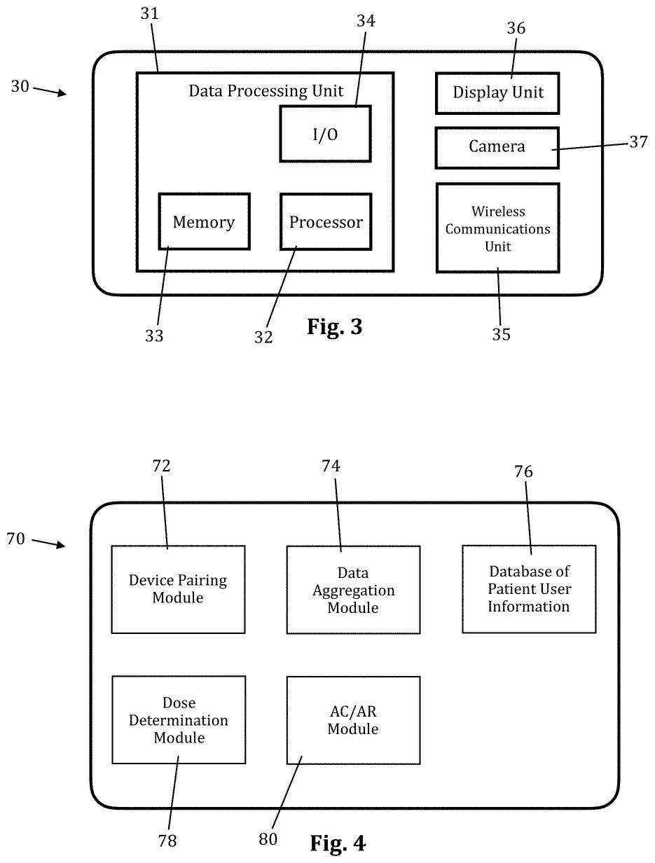

FIG. 3 shows a block diagram of an example embodiment of the companion device 30 (e.g. or “companion computing device”, or “computer”) of the intelligent medicine administering system 10. The data processing unit 31 of the companion device 30 includes a processor 32 to process data, a memory unit 33 in communication with the processor 32 to store data, and an input/output unit (I/O) 34 to interface the processor 32 and/or memory 33 to other modules, units or devices of the companion device 30 or external devices. For example, the processor 32 can include a central processing unit (CPU) or a microcontroller unit (MCU). For example, the memory 33 can include and store processor-executable code, which when executed by the processor 32, configures the data processing unit 31 to perform various operations, e.g., such as receiving information, commands, and/or data, processing information and data, and transmitting or providing information/data to another device. In some implementations, the data processing unit can transmit raw or processed data to a computer system or communication network accessible via the Internet (referred to as ‘the cloud’) that includes one or more remote computational processing devices (e.g., servers in the cloud). To support various functions of the data processing unit, the memory 33 can store information and data, such as instructions, software, values, images, and other data processed or referenced by the processor 32. For example, various types of Random Access Memory (RAM) devices, Read Only Memory (ROM) devices, Flash Memory devices, and other suitable storage media can be used to implement storage functions of the memory unit 33. The I/O 34 of the data processing unit 31 can interface the data processing unit 31 with the wireless communications unit 35 to utilize various types of wired or wireless interfaces compatible with typical data communication standards, for example, which can be used in communications of the data processing unit 31 with other devices such as the pen device 20, via a wireless transmitter/receiver (Tx/Rx) unit, e.g., including, but not limited to, Bluetooth, Bluetooth low energy, Zigbee, IEEE 802.11, Wireless Local Area Network (WLAN), Wireless Personal Area Network (WPAN), Wireless Wide Area Network (WWAN), WiMAX, IEEE 802.16 (Worldwide Interoper-ability for Microwave Access (WiMAX)), 3G/4G/LTE cellular communication methods, NFC (Near Field Communication), and parallel interfaces. The I/O 34 of the data processing unit 31 can also interface with other external interfaces, sources of data storage, and/or visual or audio display devices, etc. to retrieve and transfer data and information that can be processed by the processor 32, stored in the memory unit 33, or exhibited on an output unit of the companion device 30 or an external device. For example, a display unit 36 of the companion device 30 can be configured to be in data communication with the data processing unit 31, e.g., via the I/O 34, to provide a visual display, an audio display, and/or other sensory display that produces the user interface of the health management application 40. In some examples, the display unit 36 can include various types of screen displays, speakers, or printing interfaces, e.g., including but not limited to, light emitting diode (LED), or liquid crystal display (LCD) monitor or screen, cathode ray tube (CRT) as a visual display; audio signal transducer apparatuses as an audio display; and/or toner, liquid inkjet, solid ink, dye sublimation, inkless (e.g., such as thermal or UV) printing apparatuses, etc. Similarly, a camera 37 of the companion device 30 may be configured to be in data communication with the data processing unit 31, e.g., via the I/O 34, to enable the companion device 30 to capture digital images or video, provide the companion device 30 with an optical reader for example to extract information from QR codes, UPC bar codes, and the like.

By way of example, when a dosing event occurs (e.g., an amount of fluid is dispensed from the pen device 20), a time stamp associated with the dosing event is recorded by the processing unit of the pen device 20 (e.g., stored in the memory of the pen device 20). For example, the time stamp may be the current time or a time where a count-up timer is used. When the dose information is eventually transmitted to the health management app 40 of the companion device 30, the time stamp and/or a ‘time-since-dose’ parameter is transmitted by the pen device 20 and received by the companion device 30 and stored in the memory 33 of the data processing unit 31 of the companion device 30. In some implementations, for example, the time of the dose can be determined without the pen device 20 having to know the current time. This can simplify operation and setup of the pen device 20. In some implementations, for example, a user time is initialized on the pen device 20 from the companion device 30, in which the user time is used for dose time tracking. Using the system 10, the health management app 40 can know the time of the dose relative to the current time.

Once the companion device 30 receives the dose related information (e.g. which can include the time information and dose setting and/or dispensing information, and other information about the pen device 20 related to the dosing event), the companion device 30 stores the dose related information in memory 33, e.g., which can include among a list of doses or dosing events. In some implementations, for example, via the user interface of the health management app 40, the companion device 30 allows the patient to browse a list of previous doses, to view an estimate of current medicament active in the patient's body (“medicament on board”) based on calculations performed by a medicine calculation module of the health management app 40, and/or to utilize a dose calculation module of the software application to assist the patient regarding dose setting information on the size of the next dose to be delivered. For example, the patient may enter carbohydrates to be eaten and current blood sugar, and the companion device 30 would already know insulin on board. Using these parameters, a suggested medicine dose (e.g., such as insulin dose), calculated by the dose calculation module, may be determined. In some implementations, for example, the companion device 30 can also allow the patient to manually enter boluses into the pen device 20 or another medicine delivery device. This would be useful if the patient was forced to use a syringe, or if the battery in the pen device 20 was depleted.

Example embodiments and implementations of the disclosed intelligent medicine administering system 10, including the health management app 40 operable on a companion device 30 able to communicate with a medical device (e.g., medicine dispensing device such as a pen device 20), are described. Some examples of features of an intelligent medicine administering system that can be used with the example methods, devices and systems for providing a prescription-regulated software controls on the system are described in U.S. Pat. No. 9,672,328 B2, entitled “Medicine Administering System Including Injection Pen and Companion Device,” the entire content of which is incorporated by reference into this disclosure for all purposes.

While the disclosed embodiments described herein are primarily based on diabetes management systems and methods involving an insulin pen, health management app associated with the insulin pen, and/or glucose monitoring devices to facilitate understanding of the underlying concepts, it is understood that the disclosed embodiments can also include treatment of other health conditions using other medications by the pen device, health management app, and/or monitoring of other analytes by sensor devices.

FIG. 4 shows a block diagram of a software architecture 70 of data processing modules in accordance with some example embodiments of the health management app 40 of the intelligent medicine administrating system 10. In some embodiments, some or all of the data processing modules of the software architecture 70 of the health management app 40 are resident on the companion device 30. In some implementations of the example software architecture 70, the modules operate to control certain functionalities of the companion device 30, which includes aggregating and processing output signals transmitted by the medical injection device (e.g., pen device 20) to execute a device pairing for validation of the medicine injection device and unlocking of some or all functionality of the health management app 40 on the companion device 30.

In some embodiments of the software architecture 70, some or all of the data processing modules are resident on the companion device 30, e.g., resident in the data processing unit 31. In some embodiments of the software architecture 70 of the system 10, some of the data processing modules can be resident on the pen device 20, e.g., resident in the electronics unit. In some embodiments of the software architecture 70 of the system 10, some of the data processing modules may be resident on smart accessories configured for use with standard (non-intelligent) dispensing devices. Similarly, in some embodiments, for example, some of the data processing modules of the software architecture 70 can be embodied as part of a data processing system 50 in the cloud (e.g., resident on one or more cloud computers).

The data processing modules of the health management app 40 on the companion device 30 may be include different or the same data processing modules of the software architecture 70 resident on the pen device 20. For example, one of the data processing modules that can be resident on and implemented by the pen device 20 includes a device pairing module 72 to receive and process the output signals from the pen device 20 to pair with the health management app 40 resident on the companion device 30.

In some implementations, the pen device 20 can be paired to the companion device 30 to enable some or all protected features of the health management app 40 associated with a specific user of the pen device 20 and/or data management on the companion device 30 which require secure access for use by the patient user. The secure pairing methods ensure that the health management app 40 resident on the companion device 30 is specifically associated with a particular pen device 20 belonging to the patient user, e.g., who may have a prescription corresponding to use of some or all features of the health management app 40.

In some embodiments of the software architecture 70 for the health management app 40, the data processing modules may also be configured to process health metric data and contextual data obtained by the health management app 40 on the companion device 30, from the pen device 20, from the sensor device 60, and/or from other devices of the patient user and/or apps on the companion device 30. In some embodiments, for example, the software architecture 70 includes a data aggregation module 74 to obtain the health metric data from a device, such as the pen device 20, a sensor device 60, and/or other devices or apps in communication with the companion device 30. The software architecture 70 includes or is in communication with a database 76 to store the health data and contextual data associated with the patient user and populations of patient users. In various embodiments, the database 76 can be resident on the data processing system 60, e.g., in the cloud. In some embodiments, the software architecture 70 includes a dose determination module 78, such as a dose calculator module, to autonomously calculate a dose of the medicine associated with dose injections from the pen device 20 based on time-relevant and context or circumstances-relevant data specific to the patient user of the pen device 20. In some embodiments, the software architecture 70 includes an augmented context and augmented reality carb/dose determination module (“AC/AR Module”) 80 to determine and display the carb value and/or required mealtime insulin dose associated with a particular food item that the user is considering to consume (e.g., imminently at a later time). The example modules shown in the software architecture diagram can be organized to provide data to one another in various ways, including directly (e.g., module-to-module) and/or indirectly (e.g., via an intermediary module), based on a periodic, an intermittent, and/or a per-request basis.

There are established equations for calculating insulin doses based on the amount of carbohydrates being eaten, but often estimating the carbs within a particular food or meal is difficult and imprecise. Also, traditional dose calculators and meal estimation tools aim to assess food that has already been selected for a meal. However, providing carb or insulin information before selecting a meal or choosing where to dine could help educate patients and influence their decisions for better health and BG control.

Disclosed are methods and systems for augmented reality and contextual dose calculations for patient users managing a health condition, such as diabetes, using an interactive health management software application 40 on a companion computing device 30 (e.g. smartphone or other mobile device) configured to aggregate, process, and display data and command functionalities of the companion device 30. For example, implementations of the disclosed methods and systems are envisioned to create an augmented reality experience operated on a companion computing device 30 with the patient user's medical device (e.g., pen device 20) to simplify carb estimation and conveniently inform the patient users about food nutrition before they make a selection.

By way of example, the app 40 is operable to utilize features of the companion device 30, such as a camera 37, display screen 36, etc., to create an augmented reality experience, e.g., by overlaying new data calculated by the computer from aggregated data of the user's meal options (based on location, past meal and other information) and recommended insulin dose presently or in the future (based on tracked glucose and insulin data). Artificial intelligence may be implemented with the app 40 to optimize or otherwise enhance the functionality, such as (by way of example only) dose recommendations and/or data aggregation.

Example embodiments described herein illustrate how the app 40 can display estimated grams of carbs for a particular food option (also referred to as “carb value”), or the equivalent dose of insulin needed for the particular user based on their clinical parameters (also referred to as “insulin dose”) if the user selects a particular food option. In some implementations, the user may choose which information to regularly display (e.g. carb value or insulin dose), for example by selecting a corresponding Settings option on the companion device 30 user interface.

In some implementations, when multiple food choice items are displayed together, the app 40 may indicate visually to the user which food items are high-carb (high-insulin-required) items the and/or which food items are low-carb (low-insulin-required) items to help guide user selection of the food items. For example, for each food item, the app 40 (e.g. by way of processor 32) determines the carb value or required insulin dose, e.g. by sourcing the data from published nutritional information (brand-specific or restaurant-specific), crowd-sourced database of nutritional information, or a database of similar items food items, and the like, and then compares the determined value (carb or dose) to predetermined high and low threshold values. Upon determining that a food item for which the calculated or determined carb value or required insulin dose meets or exceeds the predetermined high threshold value, the app 40 may enhance the value displayed on the user interface with an appropriate visual indicator (e.g. red color and/or flashing, etc.) to call the user's attention to the potentially unhealthy food item. Similarly, upon determining that a food item for which the calculated or determined carb value or required insulin dose is below the predetermined low threshold value, the app 40 may enhance the value displayed on the user interface with an appropriate visual indicator (e.g. blue or green color and/or flashing, etc.) to call the user's attention to the potentially healthy food item.

In some embodiments of the app 40, carbs or insulin required may also be displayed in a relative measure, such as a color scale from red to green, or a scale of one dot to five dots.

In some embodiments, such as cases where a full meal for immediate consumption is listed, the insulin dose listed by the app 40 may include more or less insulin to correct the user's current BG level as well as cover the food. In cases where multiple items will be eaten immediately (like picking multiple things from a refrigerator to make a meal) or shopping for future meals from a grocery store, listed BG correction with each value may not be possible, so each item may simply be listed with the amount of insulin required to cover its carbs, regardless of more or less insulin the patient currently needs for correction.

Patients receiving insulin injection therapy (contrasted with an insulin pump, for example) who have to take a painful shot every time they administer an insulin bolus may benefit from implementations of the app 40 by seeing foods highlighted that would not require an insulin dose. This could be because the foods have no carbs, or because a dose calculator determines that the patient's current condition (glucose level, glucose trend, and/or insulin on board) would not require additional insulin if the food were eaten.

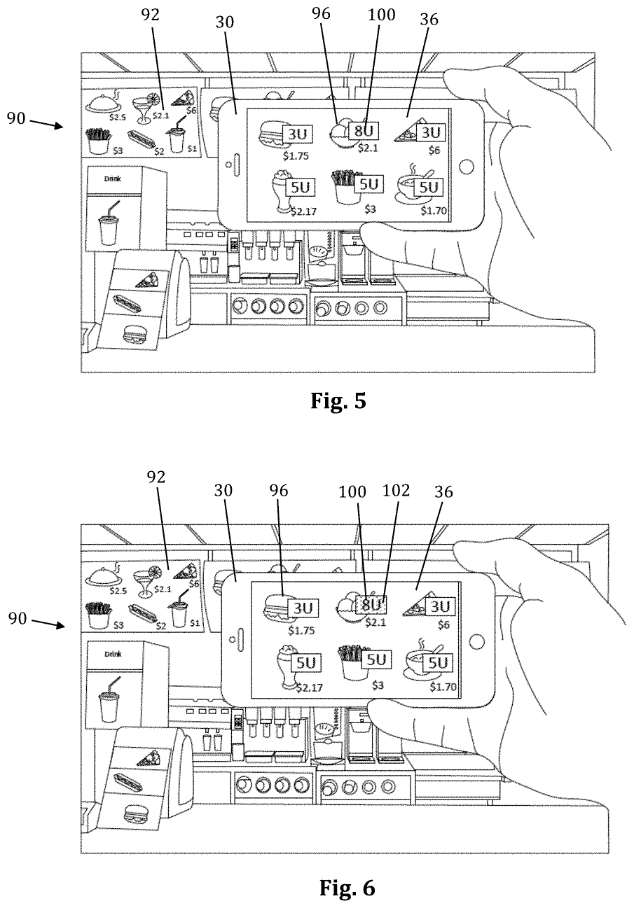

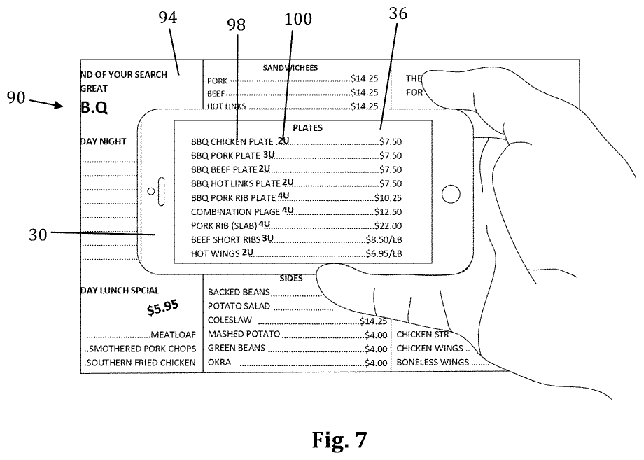

FIGS. 5-7 illustrate an example implementation of the augmented reality dose calculator module 80 in use in a restaurant environment 90. The augmented reality experience of the present example calculates and displays the dose of medicine estimated for identified food or beverage choices imaged by the user's companion device 30, in which the displayed dose calculations are overlaid or presented proximate the food or beverage choice.

In such a situation, the user orients the companion device 30 (e.g., smartphone or other mobile device) such that the camera 37 of the device is directed toward an information display within the environment 90. By way of example, the information display may be an overhead menu board 92 or a hand-held printed menu 94 with pictures 96 of food items, printed words 98 identifying the food items, or a combination of pictures and words. The camera 37 digitally transmits the images to the data processing unit 31 in the companion device 30, which then identifies the food items via text or photo recognition capabilities. The data processing unit 31 then determines the corresponding carb values and/or required insulin dose by sourcing data from published restaurant-specific nutritional information, crowd-sourced database of nutritional information, or a database of similar items food items, and the like, and causes the app 40 to display the determined carb values or required insulin as an overlay 100 on the real-time image on the display unit 36 of the companion device 30, in such a way that the user sees the carb value or required dose superimposed on the device screen 36 in real time. For example, upon viewing a menu in a restaurant through the display screen 36 and camera 37 of the companion device 30, the carb value or insulin required of each item may be superimposed next to the name or picture of each item to enhance the ability of the user to make healthy food choices.

By way of example, this feature may be accessed or activated through the app 40, by the user selecting an appropriate option or icon in the app 40, which directs the companion device 30 to access or utilize the camera 37 and activate the AC/AR dose calculation module 80. In some implementations, a GPS positioning can automatically determine the store/restaurant the user is visiting, which app 40 may prompt the user to confirm for example by way of a popup window on the user interface. Text recognition would identify the various menu items, and carb estimates could be sourced from published nutritional information for the restaurant, from crowd-sourced databases of nutritional information, or (in the case where the specific restaurant has not been cataloged) based on a database of common food dishes with similar names. As described above, the app 40 may identify food items that are high-carb or low carb and then highlight 102 those items on the user interface, for example as illustrated in FIG. 6.

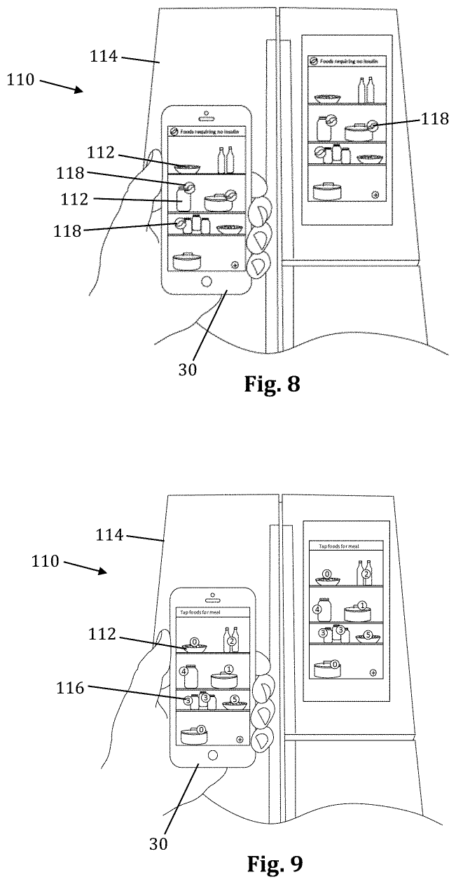

FIGS. 8-9 illustrate an example implementation of the augmented context dose calculator module 80 in use in a home environment 110. In the present example, the app 40 may create an augmented context for information about the consumable contents 112 in a user's refrigerator 114, pantry or other food/beverage storage location that provides prospective dose calculations 116 associated with the contents 112.

“Smart” refrigerators 114 exist that have a camera within them and can display contents on a display of the refrigerator or on another device such as a smartphone. By way of example, the app 40 can create the augmented experience on the companion device 30 where, on the image of the food 112, the carbs per serving and/or the required insulin dose 116 may be superimposed to inform the user and help the user make informed food choices. This process occurs in a substantially similar manner to that described above with respect to the restaurant environment 90. Notably, without a smart fridge (or for food in a pantry or other storage location in the home), a similar approach can be used with a smartphone's camera.

In some implementations, food items 112 in the refrigerator may be identified manually by the user when they are purchased and initially placed in the refrigerator (or pantry), for example by selecting the item from a catalogue or database within the app or accessible via the Internet, by using the camera 37 of the companion device 30 to scan the bar code or QR code, or via image recognition and a nutritional database.

The app 40 could also be used to quickly allow selection of foods to be eaten to calculate a total dose. For example, the user may tap the items or the carb/insulin numbers displayed on the display unit 36 of the companion device 30 to add the selected items to a “current meal” count, which the app 40 would then use to calculate the appropriate insulin dose for the full meal. In some implementations, the app 40 may overlay a unique symbol 118 on food items 112 that have a zero carb count or require no meal time insulin dose.

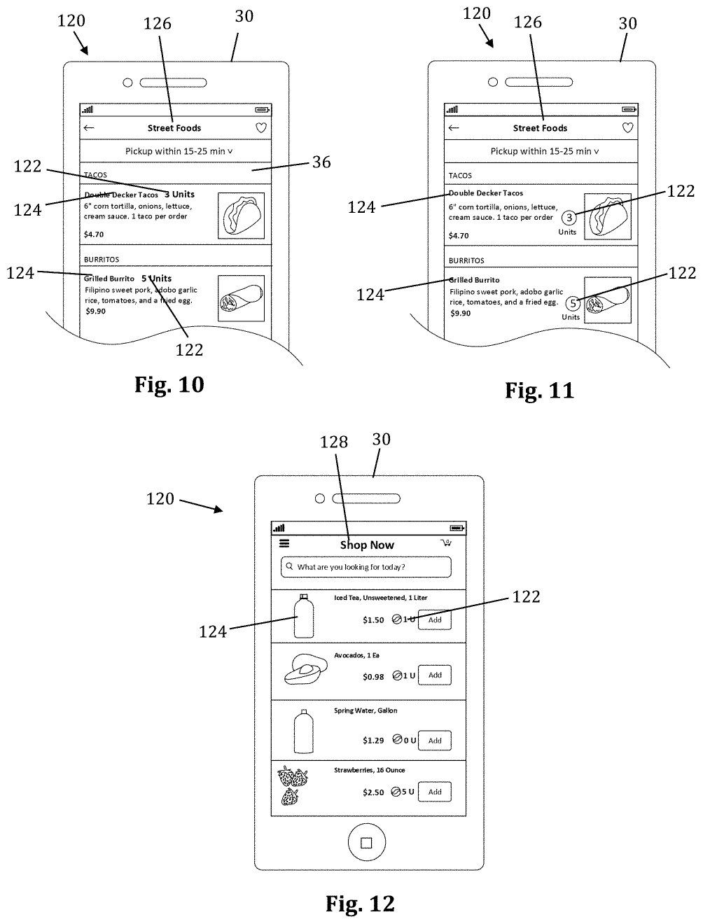

FIGS. 10-12 illustrate an example implementation of the augmented context dose calculator module 80 in use in an online food-ordering environment 120. In the present example, the app 40 may provide carb value and/or prospective dose information 122 about the foods available for ordering 124 through the use of a third party app 126 or a third party website 128. As used herein, “augmented context” means the dynamic presentation of nutritional data, dose calculations based on associated nutritional data, or metrics associated with past glucose responses to consumers via the app 40 disclosed herein, wherein the nutritional and/or glucose response data is extracted and consolidated from any number of sources and enables actionable decision making on the part of the consumer.

Popular third party apps and websites (e.g., Uber Eats, Instacart, Amazon Prime Now, Grubhub, Yelp, etc.) allow remote ordering of groceries and restaurant foods. In some embodiments, the health management app 40 is able to produce an interactive interface that can have carb/BG values 122 listed alongside the items 124 in the third party app 126 or web site 128 (e.g. as an overlay on the user interface). These listings may be used to help inform the user's selection for purchase. Upon purchasing the items 124, the app 40 may prompt the user to save the selection so that the user may quickly find the carb value or dose info at a later time when the food item will be eaten, or alternatively the app 40 may prompt the user to enter the food selection as a meal currently being eaten.

For example, in the case of selecting a meal that will be delivered later, it would not be appropriate to dose before the food arrives, and the BG correction portion of the dose may change (as the user's BG level changes) by the time the food arrives. For this reason, the user may select the meal and later alert the app 40 (e.g. through the user interface) that they are about to eat it. Alternatively, a continuous display on an app/phone/watch could indicate the current dose to take if they're about to eat the meal (which may change over time). Then when the meal arrives and the dose is taken, the meal may be logged and the dose recommendation cleared.

For example, food delivery services typically have predefined lists of foods and menus, so first-party integration can include storing the carb values for each item in a database of the disclosed system. Third-party integration (e.g., via a browser plugin, or a 3^{rd} party app or website) can identify the textual listings displayed to the user and develop an independently researched or crowdsourced database of nutritional information to overlay on the listings.

By way of example, FIGS. 10-11 illustrate an example of a restaurant takeout mobile order interface displayed as an image on the display unit 36 of the companion device 30, showing in particular a portion of the restaurant's menu or catalogue of food items available for purchase. FIG. 12 illustrates an example of a mobile order interface of an e-commerce retailer configured for a grocery order, showing a portion of the retailer's catalogue of food items available for purchase.

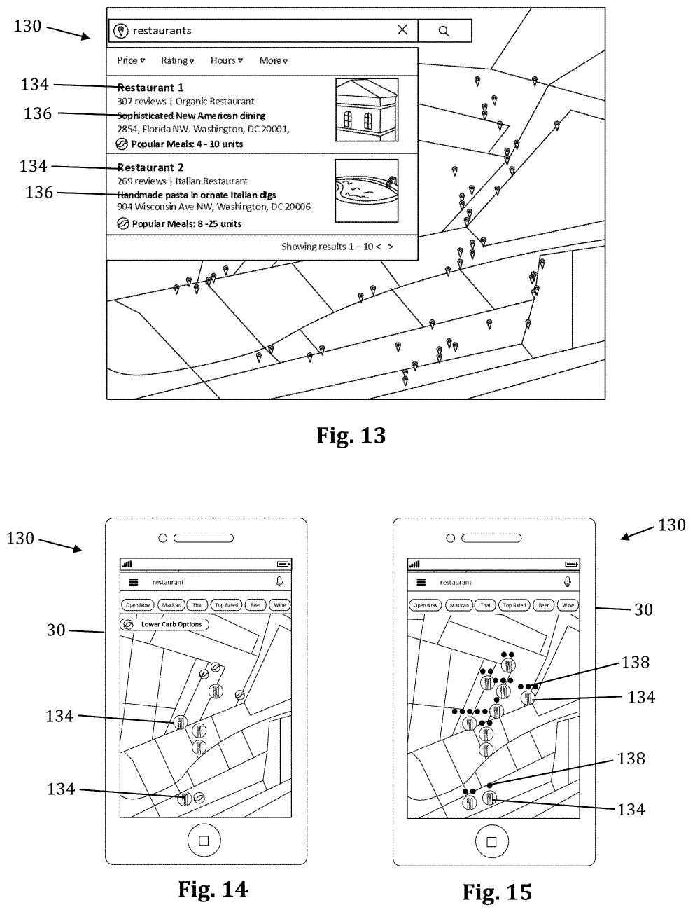

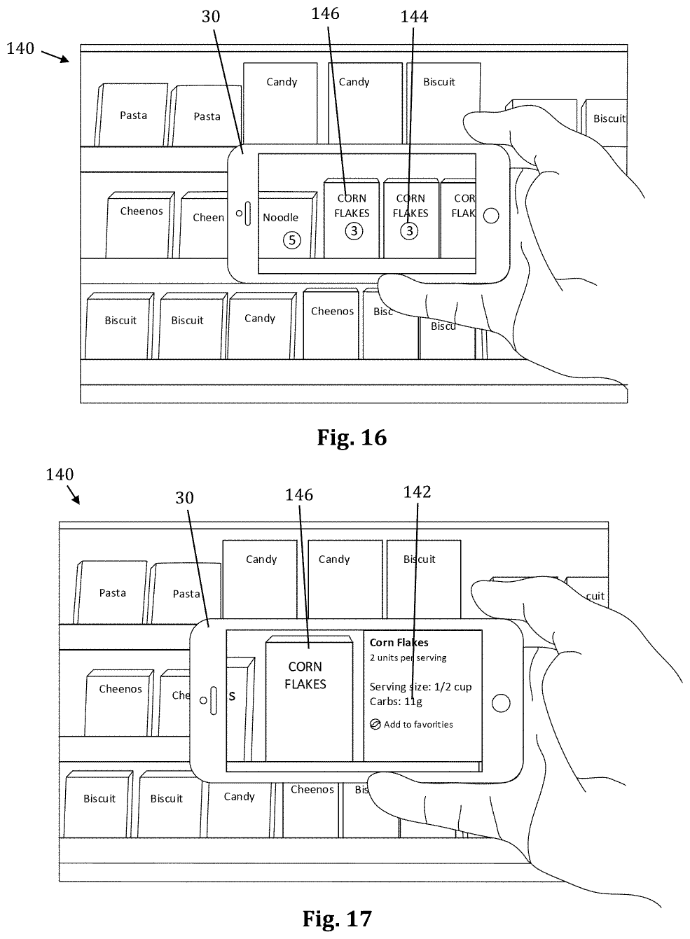

FIGS. 13-15 illustrate an example implementation of the augmented context dose calculator module 80 in use in an online map search environment 130. In the present example, the app 40 uses location information of the user and/or user content that provides prospective dose information about the foods available at the displayed venues 134.

In some implementations, the health maintenance app 40 may create an augmented context and/or reality about the information directed to the user on the user's companion device 30 by using geographical location information of the user or geographical location information of content being viewed by the user. For example, in some implementations, when a user is using a web search, mapping app, reviews site, or similar to select a location for dining, these results (in list form, and/or displayed graphically as on a map) may display corresponding carb or insulin information. This could be in the form of highlighting locations with particularly low-carb or high-carb food choices, listing the common carb values for meals eaten there, or ranking higher and lower carb options graphically.

Locations 134 or menu items 136 could also be highlighted or rated if they tend to be popular with other users of the dose calculator, or are rated positively by other users of the dose calculator. For example, a restaurant with good healthy diabetes-friendly choices (e.g. as determined by the app 40 using one or more of the processes described above) may be highlighted in a search result and or rated according to number and popularity of diabetes-friendly choices, helping educate people who may be new to an area or new to counting carbs and managing diabetes. The highlighting or rating may be based on how frequently other users of the app select this location (or food item), how much insulin is typically required for other users, the average BG excursion or time out of target range of other users, or manual rating of a location's healthy options by other users. The ratings 138 may be displayed in the list form or map form, and may take the form of a number of stars, dots and the like (for example 1-5) displayed near the listing (see, e.g. FIG. 15).

FIGS. 13-15 illustrate an example implementation of the augmented context dose calculator module 80 in use in a retail store environment 140. In the present example, the app 40 uses prepackaged food information that estimates dose information pertinent to the user in real time while displaying the estimated dose calculation (e.g. carb value 142 or required dose 144 overlaid or proximate to images of the prepackaged food 146 and/or associated information.

In some implementations, app 40 can create an augmented experience to provide estimated dose information on the user's companion device 30 based on and displayed with food information of prepackaged items 146. For example, in some implementations, when shopping at a store, the user may aim the smartphone (or similar) camera 37 at a product 146 on the shelf, or the product's barcode, or listing of nutritional information. After identifying the product or processing the nutritional information, the app 40 may then display the carbs per serving 142 and the amount of insulin required per serving 144. This could help educate the user and inform their purchasing decisions, and could also serve as a simple interface to save the items being purchased for future reference in the dose calculator. Multiple items on a shelf could be independently identified with carb and/or insulin values superimposed on them to help users quickly compare products.

Nutritional information for products could be accessed from a trusted/published database, could be from crowd-sourced information of users manually entering values, or could be aggregated over time by users—if a product is unknown in the database the user may be prompted to photograph the nutrition label so it can be correlated to the product's packaging image, packaging text, and barcode so that future users have access to this information.

This same approach could be used outside the grocery store, such as at a user's own home for products they already own.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter affecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a standalone program or as a module, component, subroutine, or other unit suitable for use in a computing environment.

A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A computer program product embodied in a non-transitory computer readable storage medium and comprising computer instructions for:
identifying at least one food item presented in an image displayed on a display unit of a user's electronic device;
determining at least one of a carbohydrate value and an equivalent dose of insulin needed for a particular user based on the user's clinical parameters for each identified food option; and
displaying, on the display unit of the user's electronic device, a visual overlay superimposed on the displayed image, the visual overlay comprising the determined at least one of carbohydrate value and equivalent dose of insulin needed for the particular user for each identified food item.

2. The computer program product of claim 1, wherein:
the user's electronic device is a camera device having a view range that is displayed on the display unit during use; and
the operation of identifying food items presented in an image displayed on a display unit comprises identifying at least one food item in the displayed view range of the camera device.

3. The computer program product of claim 2, wherein the camera device comprises a mobile communication device having a data processing unit including a processor, memory unit, and input-output unit, a display unit to present a user interface, and a camera to digitally capture images.

4. The computer program product of claim 3, wherein the computer program product is embodied in the memory unit of the mobile communication device.

5. The computer program product of claim 1, wherein the operation of identifying food items presented in an image displayed on a display unit comprises at least one of recognizing text of a restaurant menu and recognizing pictures of a prepared food item.

6. The computer program product of claim 5, wherein the operation of determining carbohydrate value comprises sourcing carbohydrate value estimates from at least one of published restaurant-specific nutritional information, crowd-sourced databases of nutritional information, and a database of common food dishes with similar names.

7. The computer program product of claim 2, wherein the operation of identifying food items in the displayed view range of the camera device comprises at least one of recognizing pictures of a raw food item, recognizing pictures of a packaged retail food item, and scanning a bar code on the packaging of a packaged retail food item.

8. The computer program product of claim 7, wherein the operation of determining carbohydrate value comprises sourcing carbohydrate value estimates from at least one of a published database of raw food nutritional information, a published database of packaged food nutritional information, nutritional information printed on retail packaging, nutritional information data obtained by scanning a bar code printed on retail packaging, and crowd-sourced databases of nutritional information.

9. The computer program product of claim 1, further comprising computer instructions for:
receiving inputs from the user to confirm consumed food choices and time of consumption;
generating a medicine dose recommendation based upon at least one of the inputted at least one identified food item, time of consumption, and user-specific clinical parameters; and
displaying, on the display unit of the user's electronic device, the generated dose recommendation.

10. The computer program product of claim 1, further comprising computer instructions for:
comparing the determined at least one carbohydrate value and required insulin dose against a predetermined high threshold value and a predetermined low threshold value;
upon determining that a food item for which the determined at least one carbohydrate value and required insulin dose at least one of meets and exceeds the predetermined high threshold value, applying an enhanced visual indicator to the displayed visual overlay to visually differentiate a high carb food item; and
upon determining that a food item for which the determined at least one carbohydrate value and required insulin dose is below the predetermined low threshold value, applying an enhanced visual indicator to the displayed visual overlay to visually differentiate a low carb food item.

11. The computer program product of claim 1, further comprising computer instructions for:
upon determining that a food item for which at least one of the determined at least one carbohydrate value and required insulin dose has a value of zero, applying an additional visual indicator to the displayed visual overlay to visually signify a no dose required food item.

12. The computer program product of claim 1, wherein the user's electronic device comprises a mobile communication device having a data processing unit including a processor, memory unit, and input-output unit, and a display unit to present a user interface.

13. The computer program product of claim 12, wherein the image displayed on the display unit of the user's mobile communication device comprises a home page published by a server and provided to the user's mobile communication device via an interface with a mobile application service provided by the server, wherein content published by the mobile application service includes the home page and a catalogue of food items available for purchase associated with the home page.

14. The computer program product of claim 13, wherein the operation of identifying food items presented in an image displayed on a display unit of a user's mobile communication device comprises recognizing text listings of the items in the catalogue of food items available for purchase.

15. The computer program product of claim 13, wherein the operation of determining carbohydrate value comprises sourcing carbohydrate value estimates from at least one of published restaurant-specific nutritional information, databases of common food dishes with similar names, published raw food nutritional information, published brand-specific packaged food nutritional information, and crowd-sourced databases of nutritional information.

16. The computer program product of claim 1, wherein the user's clinical parameters include at least one of medicament on board data and time-since-dose data.

17. The computer program product of claim 16, wherein the at least one of medicament on board data and time-since-dose data is provided by a user's pen device in electronic communication with the user's electronic device.

18. The computer program product of claim 1, wherein the user's clinical parameters are determined at least in part from data received from a user's pen device in electronic communication with the user's electronic device.
